(19) United States
(12) Patent Application Publication
(10) Pub. No.: US 2010/0324476 A1
(43) Pub. Date: Dec. 23, 2010

(54) FLUIDICS CONTROL VIA WIRELESS TELEMETRY

(76) Inventors: Mikhail Boukhny, Laguna Niguel, CA (US); John Morgan Bourne, Tustin, CA (US); Glenn Robert Sussman, Laguna Nigel, CA (US)

Correspondence Address: ALCON, IP LEGAL, TB4-8, 6201 SOUTH FREEWAY, FORT WORTH, TX, 76134, US

(21) Appl. No.: 12/486,459

(22) Filed: Jun. 17, 2009

(51) Int. Cl. A61F 9/007; A61M 31/00; G05B 23/00

(52) U.S. Cl. 604/65; 340/5.1

(57) ABSTRACT

In various embodiments, a pressure sensor may be configured to detect pressure information associated with pressure in an eye during an ophthalmic surgical procedure. The pressure information may be wirelessly communicated, through a transmitter coupled to the pressure sensor, to a receiver communicatively coupled to a surgical console. The receiver may provide the received pressure information to the surgical console to use in controlling pressure at a surgical site during the surgical procedure. For example, controlling the pressure may include maintaining a desired IOL pressure level within the surgical site at the eye. In some embodiments, the pressure sensor may include a strain gage coupled to a contact lens or a sleeve of an ocular surgical handpiece. Other pressure sensor configurations are also contemplated.

### FIELD OF THE INVENTION

The present invention generally pertains to pressure detection. More particularly, but not by way of limitation, the present invention pertains to pressure detection for fluidics control.

### DESCRIPTION OF THE RELATED ART

Various fluidic systems used in ocular surgeries today may use pressure measurements obtained from fluid lines entering and/or exiting a surgical handpiece. The pressure may be measured in these fluid lines at an external operating console. Because the fluid lines are often several feet in length, pressure losses along the length of the tubing may require intraocular (IOL) pressure levels to be extrapolated from the detected pressure in the lines. Further, there may be a response delay in obtaining IOL pressure changes because of the long fluid lines. Flexibility/compliance of the tubing may also affect a pressure determination.

### SUMMARY OF THE INVENTION

In various embodiments, a pressure sensor may be configured to detect pressure information associated with pressure in an eye during an ophthalmic surgical procedure and may wirelessly communicate this information, through a transmitter, to a receiver in communication with a surgical console. The surgical console may use the pressure information to control pressure at a surgical site during a surgical procedure on the eye. For example, controlling the pressure may include maintaining a desired IOL pressure level within the surgical site at the eye.

In some embodiments, the pressure sensor may include a strain gage coupled to a contact lens. Through the contact lens, the pressure sensor may detect changes in IOL pressure by detecting changes in comeoscleral curvature of the eye. In some embodiments, the pressure sensor may include a bridge circuit coupled to a sleeve (e.g., an irrigation sleeve) of an ocular surgical handpiece to detect a pressure of irrigation fluid within the irrigation sleeve (which may correspond to an IOL pressure of the eye). Other pressure sensor configurations are also contemplated.

### BRIEF DESCRIPTION OF THE DRAWINGS

For a more complete understanding of the present invention, reference is made to the following description taken in conjunction with the accompanying drawings in which:

FIG. 1 illustrates a block diagram of a pressure sensor for fluidics control, according to an embodiment;

FIG. 2 illustrates a pressure sensor with a strain gauge and inductor, according to an embodiment;

FIG. 3 illustrates a pressure sensor in a contact lens for an eye, according to an embodiment;

FIGS. 4a-b illustrate a pressure sensor with a bridge circuit, according to an embodiment;

FIGS. 5a-b illustrate a pressure sensor on an irrigation sleeve of a surgical handpiece;

FIG. 6 illustrates an electrical diagram of a pressure sensor with a strain gauge in communication with a fluidics system;

FIGS. 7a-c illustrate electrical diagrams of configurations of a pressure sensor with one or more strain gauges in communication with a fluidics system;

FIGS. 8a-8b illustrate embodiments of surgical consoles;

FIG. 9 illustrates a flowchart of a method for detecting pressure information, according to an embodiment; and

FIG. 10 illustrates a fluidics management system, according to an embodiment.

It is to be understood that both the foregoing general description and the following detailed description are exemplary and explanatory only and are intended to provide a further explanation of the present invention as claimed.

### DETAILED DESCRIPTION OF THE EMBODIMENTS

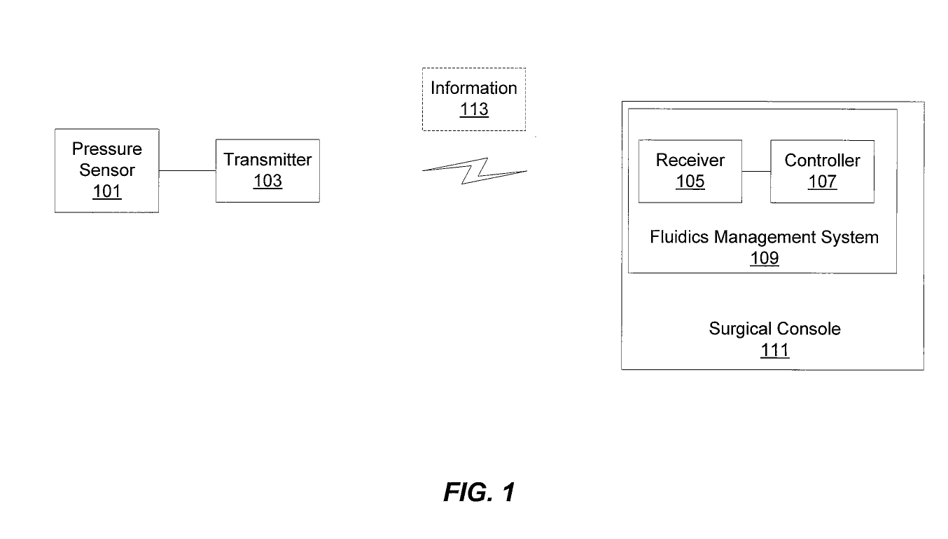

FIG. 1 illustrates a block diagram of a pressure sensor 101 for fluidics control, according to an embodiment. In some embodiments, a pressure sensor 101 may be placed near the eye (e.g., on a contact lens or a surgical handpiece sleeve) to provide pressure information 113 to a surgical system before, during, or after a surgical procedure. For example, the pressure sensor 101 may provide pressure information 113 on a detected intraocular (IOL) pressure of the eye to an ophthalmic surgical system that may then use the pressure information 113 to, for example, maintain a desired IOL pressure during the surgical procedure. Pressure sensor 101 may also provide pressure information 113 outside of a surgical context (e.g., as part of a routine eye examination).

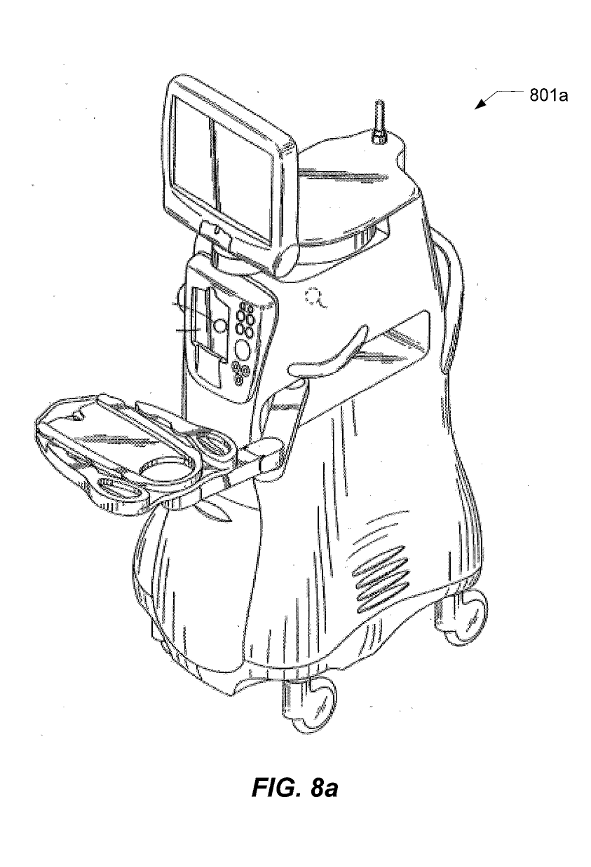

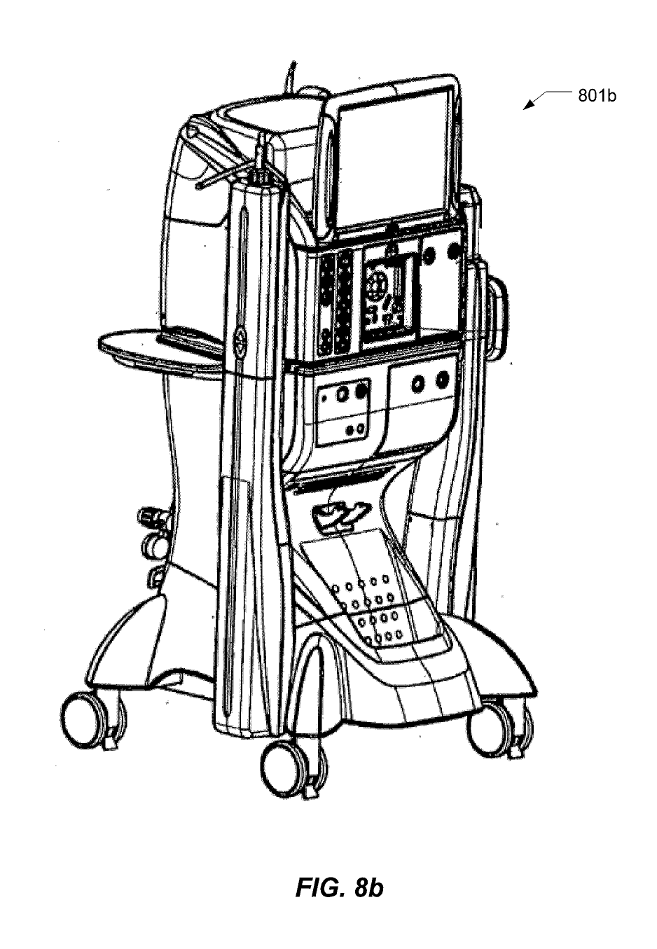

In various embodiments, pressure information 113 from the pressure sensor 101 may be wirelessly transmitted through a transmitter 103 to a receiver 105. In some embodiments, the transmitter 103 and pressure sensor 101 may be coupled together on a contact lens or surgical handpiece sleeve (e.g., through a wired or wireless connection). In some embodiments, the receiver 105 may be coupled to a fluidics management system 109 of a surgical console 111. In some embodiments, the receiver 105 and fluidics management system 109 may be communicatively coupled through a communication link (such as a wired or wireless link). In some embodiments, the receiver 105 may be a component of the fluidics management system 109. The surgical console 111 may include a phacoemulsification surgical console 801a (see FIG. 8a) or a multifunctional surgical console 801b for anterior and posterior segment ophthalmic surgeries (see FIG. 8b). Other surgical console types and surgery types are also contemplated. The fluidics management system 109 may be a component in the surgical console 801a,b or may be a separate device. In some embodiments, a controller 107 coupled to the receiver 105 and/or a controller coupled to the pressure sensor 101 may coordinate communication of the pressure information 113 (e.g., coordinate wireless communication between the transmitter 103 and the receiver 105, information formatting, error checking, etc).

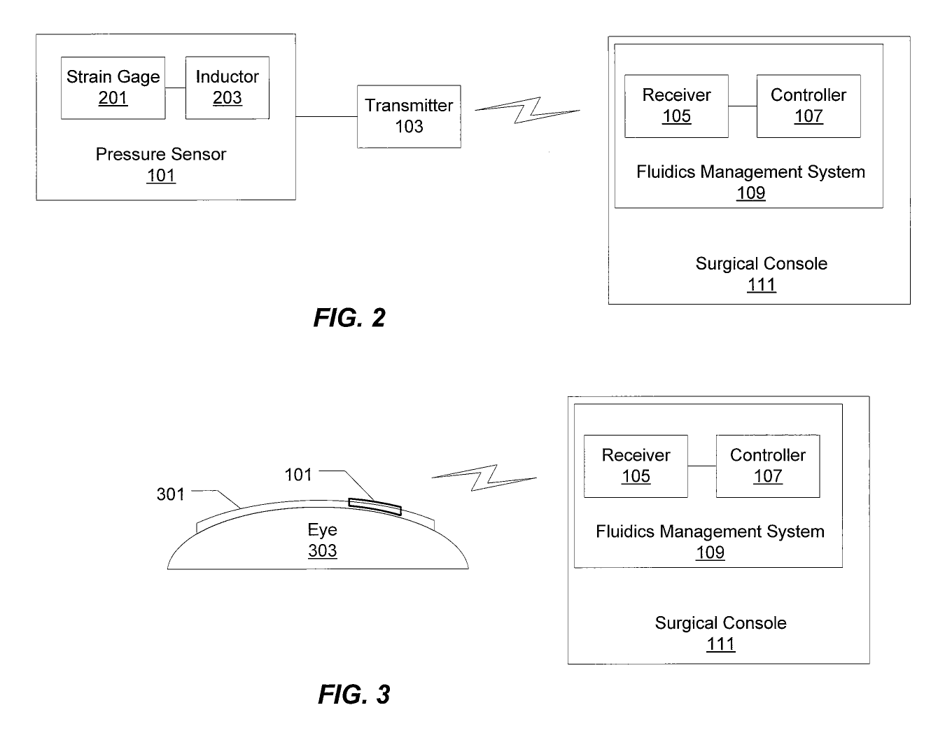

FIG. 2 illustrates a pressure sensor 101 that includes a strain gauge 201 and an inductor 203, according to an embodiment. In some embodiments, the strain gauge 201 may detect changes in forces acting on the contact lens and/or sleeve. For example, as seen in FIG. 3, a strain gauge 201 embedded in a contact lens 301 on the surface of an eye 303 may detect changes in corneoscleral curvature indicative of changes in IOL pressure. These changes may be transmitted to the fluidics management system 109 of surgical console 111 which may change one or more surgical parameters (e.g., irrigation flow rate or aspiration pressure for a handpiece) to maintain a desired IOL pressure in the eye 303. The pressure sensor 101 may be powered by the inductor 203 (e.g., wirelessly powered through an external signal received by the inductor 203). In some embodiments, magnetic telemetry may allow communication (e.g., in the form of a low frequency signal) between, for example, circuitry on the handpiece (e.g., including a primary coil) and the circuit on the pressure sensor (which may act as a secondary coil receiving and/or sending signals with pressure information back to the handpiece). Other power mechanisms and mechanism locations are also contemplated (e.g., a small lithium battery attached to the pressure sensor).

Various configurations may be used for the pressure sensor 101 embedded in the contact lens 301. For example, the pressure sensor 101 may include one or more conductive wires (e.g., in a strain gauge configuration) embedded in a soft silicone contact lens. As another example, the pressure sensor 101 may include conductive traces deposited on the surface of the contact lens 301. In some embodiments, the conductive traces may be deposited in an internal layer of the contact lens 301 (e.g., with a silicone layer above and below the conductive traces). Other materials and configurations are also possible. For example, the contact lens 301 may be made of a rigid material (e.g., glass) with a layer of less rigid material (such as soft silicone) deposited on an underside of the contact lens 301 that is configured to be in contact with the eye 303. The conductive traces may be deposited in this less rigid layer. In some embodiments, components of the pressure sensor 101, inductor 203, transmitter 103, and/or antenna may be included on a chip (e.g., a silicone chip with conductive traces) that is coupled to the surface of the contact lens 301 (or embedded inside the contact lens 301). In some embodiments, the transmitter 103 and/or antenna may be placed with the pressure sensor 101 or may be separated from the pressure sensor 101 (e.g., on a different part of the contact lens 301 or placed off of the contact lens 301). For example, the pressure sensor 101 may include a pattern of material on the contact lens 301 that is visually monitored above the pressure sensor 101 (e.g., by equipment placed external to the eye 303) for changes in the pattern (e.g., the material may be placed in lines that get closer or separate in response to changes in corneoscleral curvature). The visual monitoring may be a wireless pressure detection such that the pressure may be detected and/or communicated without wired communication between the pressure sensor 101 on the eye 303 and the surgical console 111.

In various embodiments, the pressure sensor 101 and contact lens 301 may be more directly interrelated. For example, the pressure sensor 101, inductor 203, transmitter 103, and/or antenna may be made of ultra lightweight materials (such as a thin silicone layer (i.e., the contact lens) to be placed directly on the eye 303 with thin conductive traces embedded in the silicone layer and arranged (e.g., in multiple layers similar to copper traces in a silicone wafer) to function as the pressure sensor 101, inductor 203, transmitter 103, and/or antenna). In some embodiments, the pressure sensor 101, inductor 203, transmitter 103, and/or antenna may be made up of conductive traces that are deposited directly onto the eye (e.g., by depositing a thin layer of silicone, then the conductive traces forming the circuitry of the pressure sensor 101, inductor 203, transmitter 103, and/or antenna onto the deposited layer of silicone). In some embodiments, the conductive traces may be deposited onto the eye 303 without a thin layer of silicone (e.g., the conductive traces may be sprayed onto the eye 303 using a conductive non-abrasive material that is atomized and sprayed in ultra-thin lines in an even manner to prevent sharp irregularities on the eye surface). In some embodiments, the pressure sensor 101, inductor 203, transmitter 103, and/or antenna may be inserted into the eye (e.g., through a surgical incision made in the eye during a surgical procedure) and placed on an internal component of the eye 303 (e.g., on the inside of the cornea, the anterior chamber, the iris, the posterior chamber, the sclera, the choroids, the retina, etc.) for detecting pressure on an internal structure of the eye 303. In various embodiments, the materials placed on or in the eye may be removed at a later time or may be bioabsorb able.

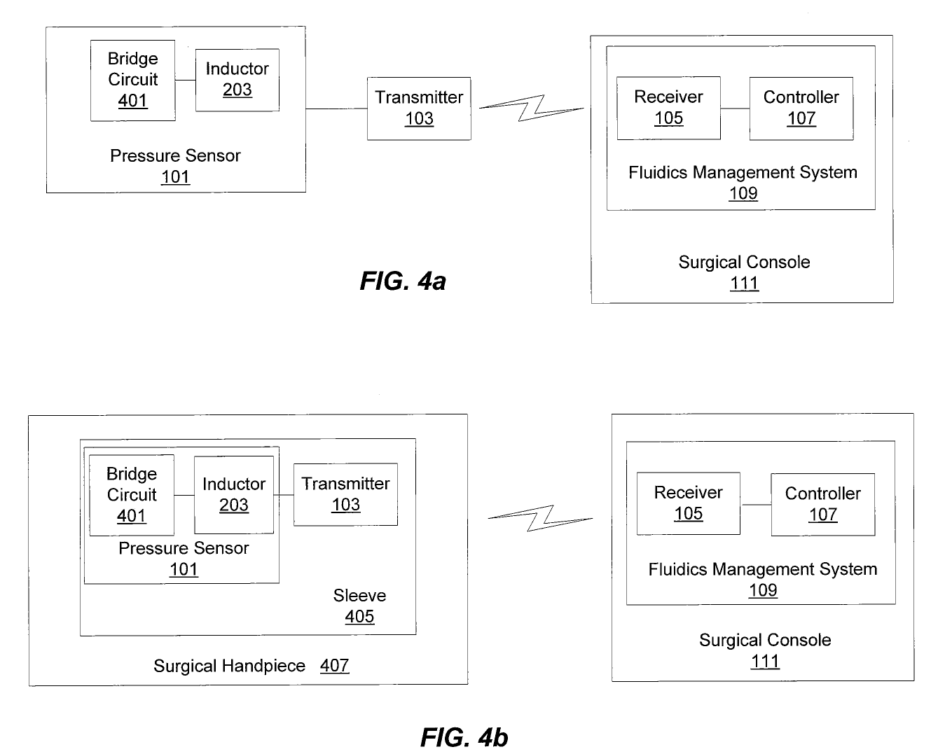

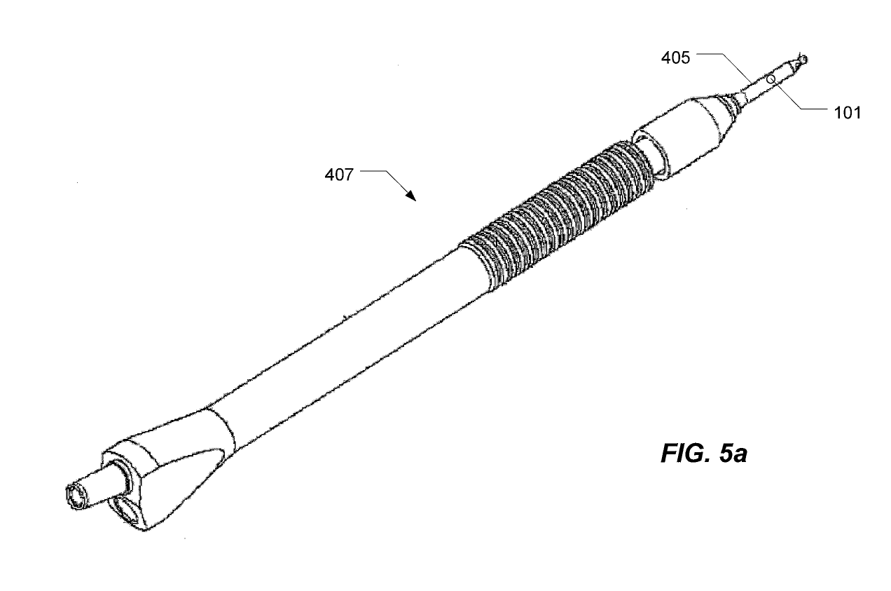

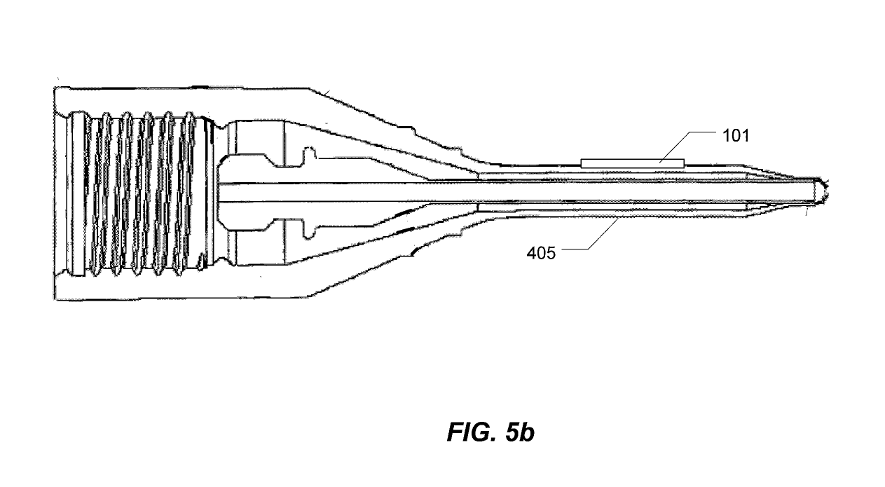

FIGS. 4a-b illustrate a pressure sensor 101 with a bridge circuit 401 that may be placed on a sleeve 405 (which may be disposable) of a surgical handpiece 407, according to an embodiment. In some embodiments, as seen in FIGS. 5a-b, pressure sensor 101 may be placed on an irrigation sleeve 405 of a phacoemulsification surgical handpiece 407. One such sleeve is described in U.S. Patent Application Publication No. 20080167604 entitled “Irrigation/Aspiration Tip” whose inventor is Karen Hong, which was filed on Jan. 9, 2007, which is hereby incorporated by reference in its entirety as though fully and completely set forth herein. Other handpiece types and sleeves are also contemplated. In some embodiments, the pressure sensor 101 may be embedded in the sleeve 405. For example, the pressure sensor 101 may include one or more conductive wires embedded in a silicone sleeve 405. As another example, the pressure sensor 101 may include conductive traces deposited on the surface of the sleeve 405. In some embodiments, the conductive traces may be deposited in an internal layer of the sleeve 405 (e.g., with a silicone layer above and below the conductive traces). Other materials and configurations are also possible. In some embodiments, components of the pressure sensor 101, inductor 203, transmitter 103, and/or antenna may be included on a chip (e.g., a silicone chip with conductive traces) that is coupled to the surface of the sleeve 405 (or embedded inside the sleeve 405). In some embodiments, the transmitter 103 and/or antenna may be placed with the pressure sensor 101 or may be separated from the pressure sensor 101 (e.g., on a different part of the sleeve 405 or placed off of the sleeve 405). A signal between the pressure sensor 101 and the transmitter may travel along a wire or conductive trace along the sleeve to the transmitter which may be on a main body of the handpiece 407.

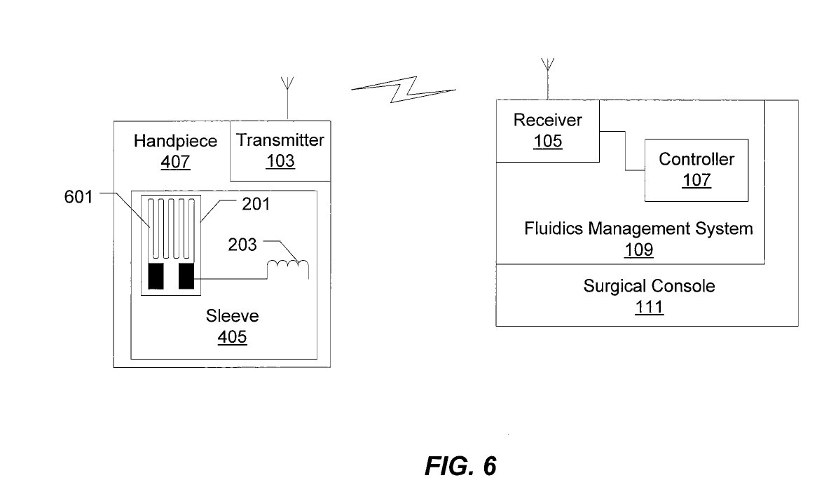

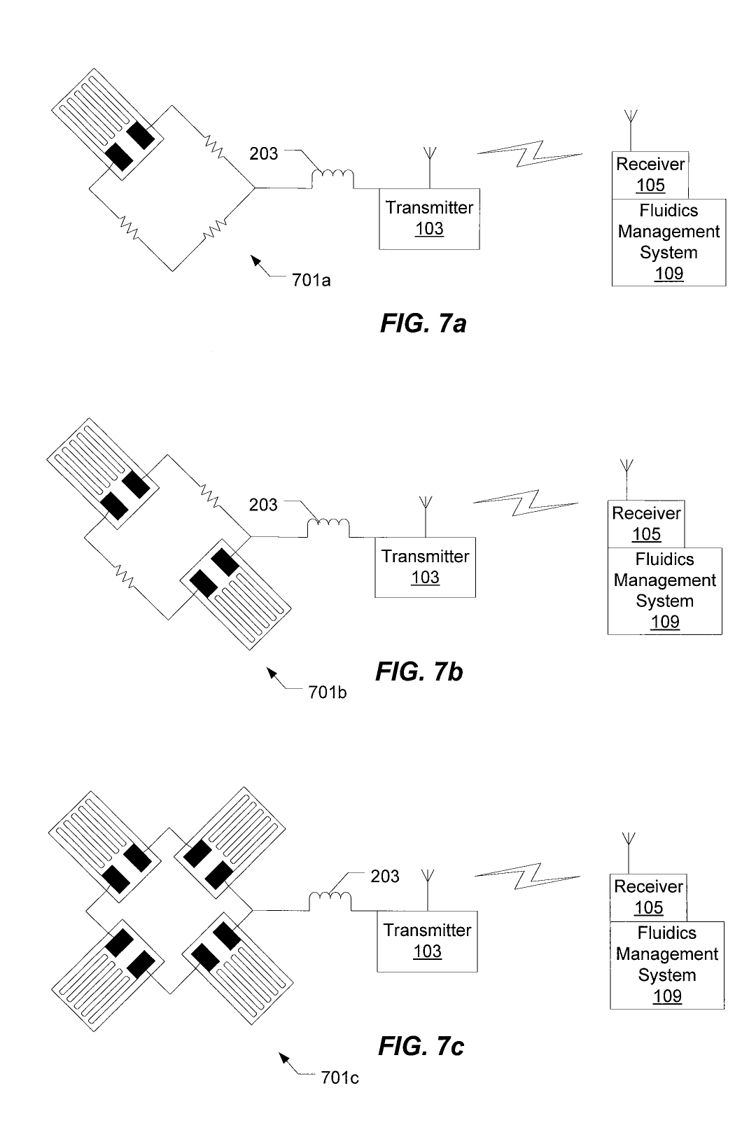

In some embodiments, the pressure sensor 101 on the irrigation sleeve 405 may detect a pressure inside the eye 303 when the sleeve is inserted in the eye 303. For example, the pressure sensor 101 on the irrigation sleeve 405 may detect a pressure of the irrigation fluid in the sleeve 405 that may be in fluid communication with the interior of the eye 303 (and, therefore, the detected pressure of the irrigation fluid may correspond to the pressure inside the eye 303). Other pressure information 113 may also be detected. The detected pressure information 113 may be communicated through a transmitter 103 to the fluidics management system 109 of a surgical console 111. As seen in FIG. 6, the pressure sensor 101 may include a strain gauge 201 and an inductor 203. In some embodiments, the strain gauge 201 may include multiple loops 601 of conductive wires and/or traces that change resistance in response to changes in underlying pressure that constrict or expand the surface of the strain gauge 201. The change in resistance (which may be indicative of the change in underlying pressure) may be detected and communicated to the fluidics management system 109. As seen in FIGS. 7a-c, the pressure sensor 101 may include one or more strain gauges 201 and resistors formed in a bridge circuit (e.g., see bridge circuits 701a-c in FIGS. 7a-c). FIG. 7a shows a single strain gauge with three resistors in a bridge circuit 701a. FIG. 7b shows two strain gauges and two resistors in a bridge circuit 701b, and FIG. 7c shows four strain gauges in a bridge circuit 701c. Other numbers of strain gauges and other bridge circuit configurations are also possible. Each additional strain gauge may add sensitivity to the bridge circuit 401 (e.g., sensitivity to detect underlying pressure changes with greater accuracy).

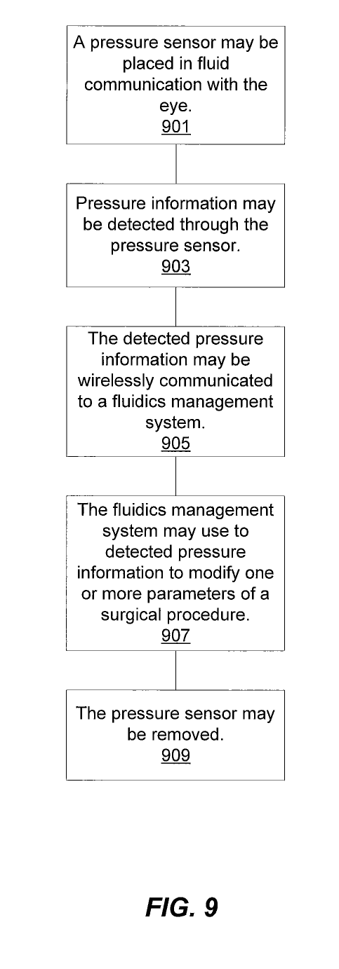

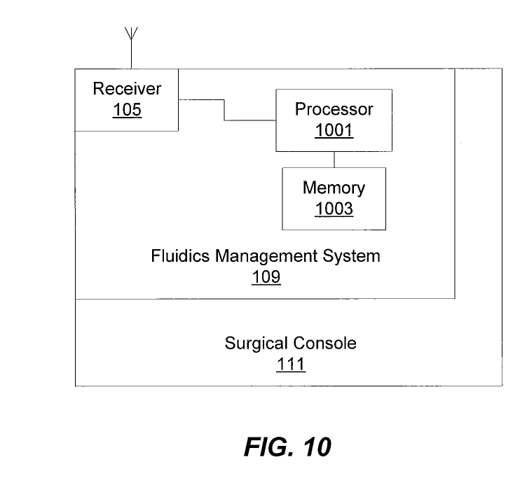

FIG. 9 illustrates a flowchart of a method for detecting a pressure associated with the eye and using the detected pressure to modify a surgical parameter. The elements provided in the flowchart are illustrative only. The provided elements may be omitted, additional elements may be added, and/or various elements may be performed in a different order than provided below.

At 901, a pressure sensor 101 may be placed in fluid communication with the eye 303. For example, the pressure sensor 101 may be placed on a contact lens 301 on the eye 303, placed on or in the eye 303, placed on a handpiece sleeve 405 that is inserted into the eye 303, etc.

At 903, pressure information 113 may be detected through the pressure sensor 101. Pressure information 113 may include a detected pressure (e.g., in mmHg) or may be information associated with pressure (e.g., a detected resistance of a strain gauge, a current level (in amps), a voltage (in volts), etc. of the pressure sensor 101). In some embodiments, the IOL pressure of the eye 303 may be detected, a pressure of an irrigation fluid in a handpiece 407 may be detected, a pressure of an aspiration line of the handpiece 407 may be detected, etc. Other information indicative of a pressure or a change in pressure is also contemplated.

At 905, the detected pressure information 113 may be wirelessly communicated to the fluidics management system 109 (e.g., on an ophthalmic surgical console). For example the detected pressure information 113 may be transmitted through transmitter 103 coupled to the pressure sensor 101 and may be received by receiver 105 in the fluidics management system 109. In some embodiments, the receiver 105 may be remote from the fluidics management system 109 and may transmit the pressure information 113 to the fluidics management system 109 through a wired or wireless interface. For example, the transmitter 103 may be embedded in a contact lens 301 or sleeve 405 and the receiver 105 may be in a handpiece 407 or other piece of surgical equipment. The receiver 105 may include a larger power source (than coupled to transmitter 103) and may be able to transmit the pressure information 113 over a greater distance to the fluidics management system 109 than the distance between the transmitter 103 and the receiver 105.

At 907, the fluidics management system 109 may use the detected pressure information 113 to modify one or more parameters of a surgical procedure being performed on the eye 303. For example, the fluidics management system 109 may adjust the irrigation flow rate or aspiration vacuum of the handpiece 407 to maintain a detected IOL pressure within a predetermined safe range (e.g., approximately between 13 to 20 mmHg) (other pressures are also contemplated). In some embodiments, the fluidics management system 109 may adjust parameters of the surgical procedure with other objectives (e.g., to maintain an aspiration vacuum level within a predetermined range). In some embodiments, the pressure information 113 may be communicated to the fluidics management system 109 in real time during the ocular surgery. In some embodiments, the pressure information may be saved and relayed in batches or upon being queried by the fluidics management system 109.

At 909, the pressure sensor 101 may be removed from fluid communication with the eye 303. For example, the contact lens 301 may be removed from the eye 303 or the sleeve 405 may be removed from the eye 303. Other removal methods are also contemplated (e.g., the pressure sensor 101 may be bioabsorbable and may be absorbed by the body). In some embodiments, the pressure sensor 101 may stay in fluid communication with the eye 303 after surgery. For example, pressure information 113 may continue to be transmitted after the ocular surgery to monitor the eye 303 for spikes in IOL pressure.

In some embodiments, the surgical console 111, fluidics management system 109, controller 107, pressure sensor, etc. may include one or more processors (e.g., processor 1001) and/or one or more memory components. The processor 1001 may include single processing devices or a plurality of processing devices. Such a processing device may be a microprocessor, controller (which may be a micro-controller), digital signal processor, microcomputer, central processing unit, field programmable gate array, programmable logic device, state machine, logic circuitry, control circuitry, analog circuitry, digital circuitry, and/or any device that manipulates signals (analog and/or digital) based on operational instructions. The memory 1003 coupled to and/or embedded in the processors 1001 may be a single memory device or a plurality of memory devices. Such a memory device may be a read-only memory, random access memory, volatile memory, non-volatile memory, static memory, dynamic memory, flash memory, cache memory, and/or any device that stores digital information. Note that when the processors 1001 implement one or more of its functions via a state machine, analog circuitry, digital circuitry, and/or logic circuitry, the memory 1003 storing the corresponding operational instructions may be embedded within, or external to, the circuitry comprising the state machine, analog circuitry, digital circuitry, and/or logic circuitry. The memory 1003 may store, and the processor 1001 may execute, operational instructions corresponding to at least some of the elements illustrated and described in association with FIG. 9.

Various modifications may be made to the presented embodiments by a person of ordinary skill in the art. For example, although some of the embodiments are described above in connection with strain gauge pressure sensors it can also be used with other types of pressures sensors and in other environments (e.g., on a blood vessel, etc). Other embodiments of the present invention will be apparent to those skilled in the art from consideration of the present specification and practice of the present invention disclosed herein. It is intended that the present specification and examples be considered as exemplary only with a true scope and spirit of the invention being indicated by the following claims and equivalents thereof.

1. A system, comprising:
a pressure sensor configured to detect pressure information associated with pressure in an eye during an ophthalmic surgical procedure;
a transmitter coupled to the pressure sensor configured to wirelessly communicate the pressure information;
a receiver configured to receive the wirelessly transmitted pressure information;
wherein the receiver is configured to provide the received pressure information to a surgical console and wherein the surgical console is configured to at least partially use the pressure information to modify one or more parameters of a surgical procedure.

2. The system of claim 1, wherein modifying one or more parameters comprises modifying an irrigation pressure or aspiration vacuum to maintain a desired intraocular (IOL) pressure level within the surgical site at the eye.

3. (canceled)

4. The system of claim 1, wherein the pressure sensor is configured to detect changes in IOL pressure by detecting changes in corneoscleral curvature of the eye.

5. The system of claim 1, wherein the pressure sensor comprises a bridge circuit coupled to an irrigation sleeve of an ocular surgical handpiece.

6. The system of claim 5, wherein the pressure sensor is configured to detect a pressure of irrigation fluid within the irrigation sleeve and wherein the pressure of the irrigation fluid corresponds to an IOL pressure of the eye.

7. The system of claim 1, further comprising an inductor coupled to the transmitter, wherein the inductor is configured to supply power to the transmitter for wirelessly transmitting the pressure information.

8. (canceled)

9. (canceled)

10. A method, comprising:
placing a pressure sensor in fluid communication with an eye;
detecting pressure information through the pressure sensor;
wirelessly communicating the detected pressure information to an ophthalmic surgical console; and
modifying at least one parameter of an ophthalmic surgical procedure using the detected pressure information.

11. The method of claim 10, further comprising removing the pressure sensor.

12. The method of claim 10, wherein modifying at least one parameter comprises modifying an irrigation pressure or aspiration vacuum to maintain a desired intraocular (IOL) pressure level within the surgical site at the eye.

13. (canceled)

14. The method of claim 10, wherein the pressure sensor comprises a plurality of traces deposited directly onto a component of the eye and wherein the method further comprises visually monitoring the traces to detect changes in IOL pressure by detecting changes in corneoscleral curvature of the eye.

15. The method of claim 10, wherein the pressure sensor comprises a bridge circuit coupled to an irrigation sleeve of an ocular surgical handpiece.

16. The method of claim 15, wherein detecting pressure information comprises detecting a pressure of irrigation fluid within the irrigation sleeve and wherein the pressure of the irrigation fluid corresponds to an IOL pressure of the eye.

17. The method of claim 10, further comprising receiving power for the wireless communication from an inductor, wherein the inductor is configured to supply power to a transmitter for wirelessly transmitting the pressure information.

18. (canceled)

19. The method of claim 10, wherein the pressure sensor comprises a silicone contact lens with at least on strain gauge comprising a conductive trace embedded in the contact lens.

20. The system of claim 1, wherein the pressure sensor and transmitter are comprised in plurality of deposited conductive traces.

21. The system of claim 20, wherein the conductive traces are deposited in a contact lens.

22. The system of claim 20, wherein the conductive traces are deposited directly on a component of the eye.

23. The system of claim 22, wherein the conductive traces are bioabsorbable.

24. The system of claim 20, wherein the plurality of deposited traces further include an inductor configured to power the transmitter.

25. The system of claim 1, wherein the receiver is on a surgical handpiece and wherein the receiver is further configured to wirelessly transmit the pressure information to a second receiver on the surgical console.
